(19) United States
(12) Patent Application Publication
(10) Pub. No.: US 2018/0325742 A1
(43) Pub. Date: Nov. 15, 2018

(54) BAND FOR COMPRESSING OR SUPPORTING A JOINT

(71) Applicant: MILLET INNOVATION, Loriol Sur Drome (FR)

(72) Inventor: Damien MILLET, Valence (FR)

(73) Assignee: MILLET INNOVATION, Loriol Sur Drome (FR)

(21) Appl. No.: 15/774,250

(22) PCT Filed: Oct. 12, 2016
(86) PCT No.: PCT/FR2016/052630

§ 371 Date: May 7, 2018

(30) Foreign Application Priority Data
Nov. 6, 2015 (FR) 1560643

(51) Int. Cl. A61F 13/06; A61F 5/01

(52) U.S. Cl. CPC A61F 13/066; A61F 5/0118; A61F 5/0111; A61F 5/0106

(57) ABSTRACT

A supporting or compressing device configured to surround a limb or a joint of a user, including an elastic band having two band segments having different stiffness values, the elastic band having an inner face in contact with an area covered by the band and an outer face opposite to the inner face, the inner face of the band providing, in the presence of a compression pressure exerted when the band is fitted under traction around a limb or joint, an adhesion with the covered area, such that the two segments of the band conserve elongations resulting from different traction forces, after removal of the traction forces.

### FIELD

The present invention relates to an articular compression or supporting device that compresses certain limited areas of the body, especially in cases of ligament or muscle weakness, or circulatory pathology, or post-operative situations. The present invention is in particular applicable to the limbs and joints of the knee, ankle, shoulder, elbow, wrist, and hips.

### BACKGROUND

Compression bands exist that are intended to be used in post-operative situations of the lower limbs, in particular to limit the formation of hematomas, and/or for circulatory pathologies, or to prevent risks of damage to the muscle or joint tissues. Dorsal support bands exist that are designed to wrap around the waist or bust forming several overlapping turns. These bands are generally made of elastic fabric, most often knitted, with an adjustable clamping device and a marking for adjusting the tightness according to the recommendations of the practitioner. These bands exert an equal pressure over the entire covered area. It also turns out that these bands tend to slide along the limb.

However, in several areas of the body it may be necessary to exert pressure locally while avoiding sensitive areas. Thus, in the example of the foot, three areas are particularly sensitive to pressure. These are the dorsal arch of the foot, the tibial peri-malleolar zone and the Achilles tendon area. Indeed, excessive pressure on these areas can cause pain or trauma. It should also be noted that the dorsiflexion of the ankle (upwards pivoting of the foot) necessary for engaging a step, is generated by the contraction of the levator muscles of the foot (anterior tibial, short and long extensors of the toes). The contraction of these muscles induces tendon bulging, especially that of the anterior tibial muscle. Too much pressure exerted in such a bulging muscle tendon area can therefore cause unbearable pain and potentially tendinopathy. In addition, too much compression of the arteries and nerves (especially the pedis artery and nerves located on the dorsal arch of the foot) can also cause a tissue oxygenation deficiency and paresthesia. In the peri-malleolar region of the tibia, excessive tightening can also have consequences on the blood circulation. Compression bands do not allow exertion of sufficient pressure on the areas to be maintained while avoiding exertion of pressure on the sensitive areas.

There are devices in the form of bands whose adjustment and locking are provided by loop and hook elements (Velcro®). Also, such a band is unable to exert pressures that are variable and adjustable depending on the covered area. Moreover, in addition to their unattractive appearance, the elements with loops and hooks are relatively thick and rigid, which can be inconvenient to the point of hindering movement, especially if the band is worn on a joint and inside a shoe. The thickness of the loop and hook elements can also hinder or prevent the wearing of a tight garment. In addition, the garments worn over the band may be damaged by the hook element whose hooks may catch on the mesh or knitting of the fabric forming the garment.

The adjustment of the pressure exerted by such a band is also difficult with loop and hook elements, because it cannot be progressive. Indeed, to change the pressure, it is necessary to completely separate the two elements and join them again in another location. Generally, the user must readjust the pressure multiple times before achieving the desired adjustment.

There are also devices in the form of socks or knitted sleeves, whose knitting is achieved selectively in rings, to locally exercise different pressures depending on the annular area covered. These devices may therefore have a relatively large thickness locally that may prevent the wearing of a tight garment. These devices also have the disadvantage of requiring the manufacture of a large number of different sizes to adapt to the different morphologies of users.

There are also non-elastic or low elasticity adhesive bands, designed to be wound in several turns around a limb to partially immobilize a joint or compress a muscle, especially in a sports or post-operative context. These adhesive bands also have the disadvantage of exerting a uniform pressure throughout the covered areas. In addition, they are necessarily for single use.

It is therefore desirable to provide a device for supporting a joint or compressing part of a limb, which can selectively exercise locally adjustable pressures depending on the area covered. It may also be desirable that this device be usable several times and be adaptable to various morphologies without requiring the provision of a large number of different sizes.

### SUMMARY

Embodiments relate to a supporting or compressing device configured to surround a limb or a joint of a user, comprising an elastic band having two band segments having different stiffness values, the elastic band having an inner face in contact with an area covered by the band and an outer face opposite to the inner face, the inner face of the band providing, in the presence of a compression pressure exerted when the band is fitted under traction around a limb or joint, an adhesion with the covered area, such that the two segments of the band conserve elongations resulting from different traction forces, after removal of the traction forces.

According to an embodiment, a first of the two band segments comprises a pad made of an elastic material, attached by two opposite ends to the band,

According to an embodiment, the pad is attached to the inner face of the supporting device.

According to an embodiment, the pad is attached to the outer face of the supporting device.

According to an embodiment, the pad comprises a layer of viscoelastic material and a layer of elastic fabric attached to the layer of viscoelastic material.

According to an embodiment, the elastic fabric layer has a lower stiffness in the longitudinal direction of the band than in the transverse direction.

According to an embodiment, a first of the two band segments has a stiffness between 2 and 4 times that of a second of the two band segments.

According to an embodiment, the inner face is formed by a layer of a polymer gel such as Polydimethylsiloxane.

According to an embodiment, the elastic band is configured in the form of a sleeve.

According to an embodiment, the inner face of the band has, in the presence of a compression pressure exerted when the band is placed under traction around a member or a joint, an adhesion with the covered area which can retain elongations resulting from stretching forces that differ by 20 N.

According to an embodiment, the device comprises first and second band portions configured to be wound around the ankle and foot, the first band portion including a first band segment having a higher stiffness than another band segment of the first band portion, the second band portion having an end fixed to the outer face of the first portion, a second band segment having a higher stiffness than another band segment of the second band portion, wherein the first and second band segments of higher stiffness are arranged on the band portions so that they can simultaneously cover the internal malleolus and regions below and above the external malleolus of the ankle.

Embodiments may also relate to a method of manufacturing a supporting or compressing device as previously defined, configured to surround a limb or joint of a user.

Embodiments may also relate to a method of using a supporting device as previously defined, the method comprising the steps of: fitting the supporting device around a limb or a joint, respectively applying a higher stretching force to a first one of the band segments having a higher stiffness than a second one of the band segments of the supporting device, and a lower stretching force to the second band segment of the supporting device, to elongate the first and second segments, and removing the stretching forces.

According to an embodiment, the supporting device comprises first and second band portion configured to be wound around the ankle and the foot, the first band portion including a first band segment having a higher stiffness than another band segment of the first band portion, the second band portion having an end attached to the outer face of the first portion, a second band segment having a higher stiffness than another band segment of the second band portion, the method comprising steps of: attaching a first end of the first band portion to an ankle orthosis having two rigid pads held on the malleoli of the user's ankle, winding, with a first traction force, the band portion around the ankle, then around the foot passing over the top of the foot, then under the foot meeting one end of the first band segment, winding the first band segment over the foot, then around the ankle exerting a second traction force greater than the first traction force, to stretch the first band segment, so as to clamp between them a lower part of a first of the two pads, and a second of the two pads, winding the end of the first band portion around the ankle with a traction force lower than the second traction force, and attaching a second end of the first band portion, winding with a traction force lower than the first traction force, the second band portion over the top of the foot, and then around the ankle, the second band segment being placed over an upper portion of the first pad, and attaching a free end of the second band portion.

### BRIEF DESCRIPTION OF DRAWINGS

Exemplary embodiments of the invention will be described in the following, without limitation in connection with the attached figures among which:

FIGS. 1 and 2 schematically show a cross-section view and a front view of a supporting device according to an embodiment,

FIG. 3 schematically shows a cross-section view of a supporting device according to another embodiment,

FIGS. 4 and 5 are schematic cross-section and front views of a supporting device according to another embodiment,

FIG. 6 schematically shows a cross-section view of a supporting device according to another embodiment,

FIGS. 7 and 8 are variation curves of the stretching force and the stiffness as a function of the elongation of elements forming the supporting device,

FIGS. 9A, 9B are schematic cross-sectional views of the supporting device fitting a limb or a joint, in two different configurations,

FIG. 10 schematically shows the external lateral face of a foot and ankle, fitted with an ankle orthosis according to an embodiment,

FIG. 10A shows a thermoformable part of the ankle orthosis of FIG. 10, according to an embodiment,

FIG. 11 schematically shows the internal lateral face of the foot and the ankle, fitted with the orthosis shown in FIG. 10,

FIG. 11A shows another thermoformable part of the ankle orthosis of FIG. 10, according to an embodiment,

FIG. 12 schematically shows a cross-section of a supporting band according to an embodiment,

FIG. 13 is a variation curve of the stretching force applied to the supporting band of FIG. 12 as it is wrapped around the foot and the ankle orthosis.

### DESCRIPTION OF EMBODIMENTS

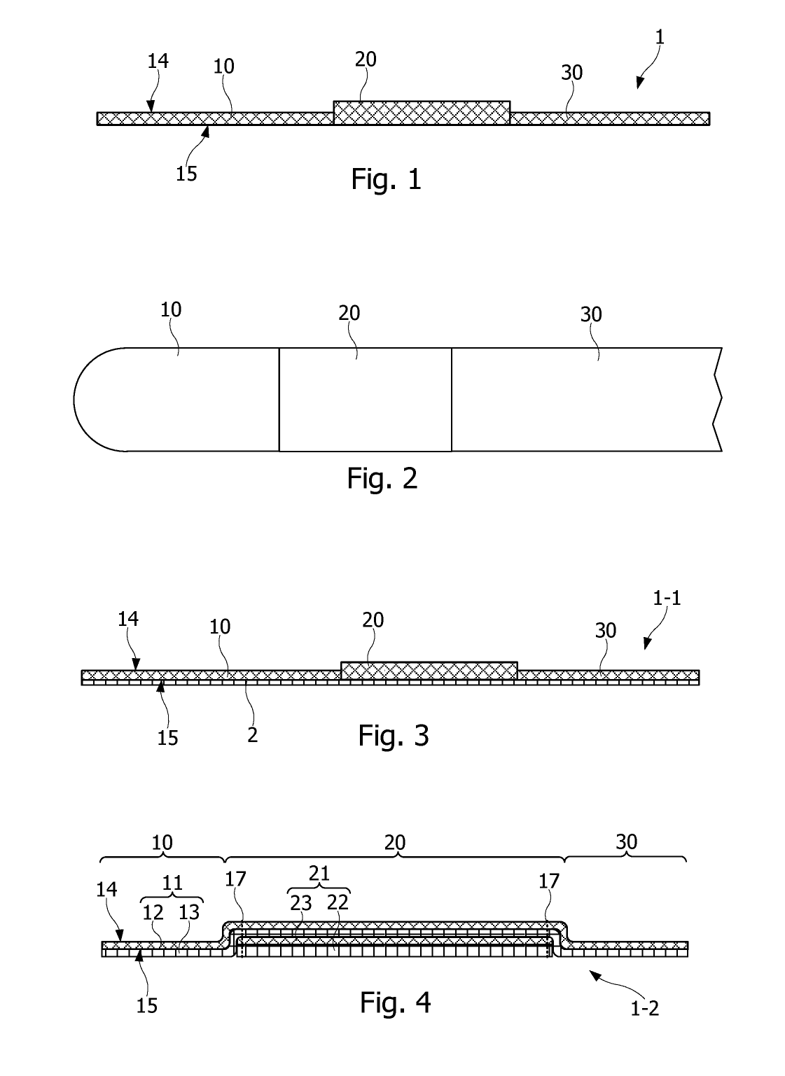

FIGS. 1 and 2 show a supporting device 1 according to an embodiment. The supporting device 1 is in the form of a band including several elastic band segments 10, 20, 30, having different stiffness values. For this purpose, the band segments 10, 20, 30 may have different thicknesses, and/or be formed in different materials. The band segments 10, 20, 30 may be joined together by any means, such as by welding or sewing.

The supporting device 1 has an outer face 14 and an inner face 15 opposite the face 14 and designed to contact the area covered by the supporting device. According to an embodiment, the inner face 15 has a certain adhesion with the covered area under the effect of pressure exerted when the supporting device 1 is placed under traction around a limb or a joint. This adhesion with the covered area is such that the band segments 10, 20, 30 can exert different pressures. These different pressures are obtained by individually subjecting to different stretching forces the different band segments 10, 20, 30, which at least partly retain their elongation after the removal of these forces due to the adhesion of the internal face 15. This adhesion depends on the contact surface, the curvature thereof (when wound around a limb), and the nature of the material on the inner face 15 of each of the band segments 10, 20, 30, as well as the material of the area covered by the device. Thanks to this adhesion, it is possible to obtain anchoring areas between which a higher compression can be exerted.

Thus, the supporting device 1, when it is fitted around a limb or a joint, can exert a higher pressure on the areas of the limb covered by the band segment 20, and, conversely, lower pressures on the areas of the limb covered by segments 10, 30.

In the example of FIG. 1, the band segment 20 has a higher stiffness than the band segments 10, 30. It can therefore be elongated under the effect of a relatively large stretching force while elongating less than the segments 10, 30 if they were subjected to the same stretching force. According to an exemplary embodiment, the band segment 20 has a stiffness between 2 and 4 times the stiffness of the band segments 10, 30. As a result, the band segment 20 extends between 2 and 4 times less than the band segments 10, 30, under the effect of the same stretching force.

According to an embodiment, the adhesion of the inner face 15 with the supporting device 1 is also obtained by a suitable choice of materials in which the band segments 10, 20, 30 are formed. According to another embodiment, this adhesion is obtained by covering the inner face 15 of the device 1 with an elastic layer in a material having the desired adhesion. The choice of materials forming the different band segments is thus not limited by an adhesion criterion. Thus, FIG. 3 shows a supporting device 1-1 including an elastic layer 2 covering at least partially its inner face 15 and presenting the desired adhesion. The layer 2 can be a coating of a viscoelastic material distributed over the inner face 15, for example with a uniform thickness.

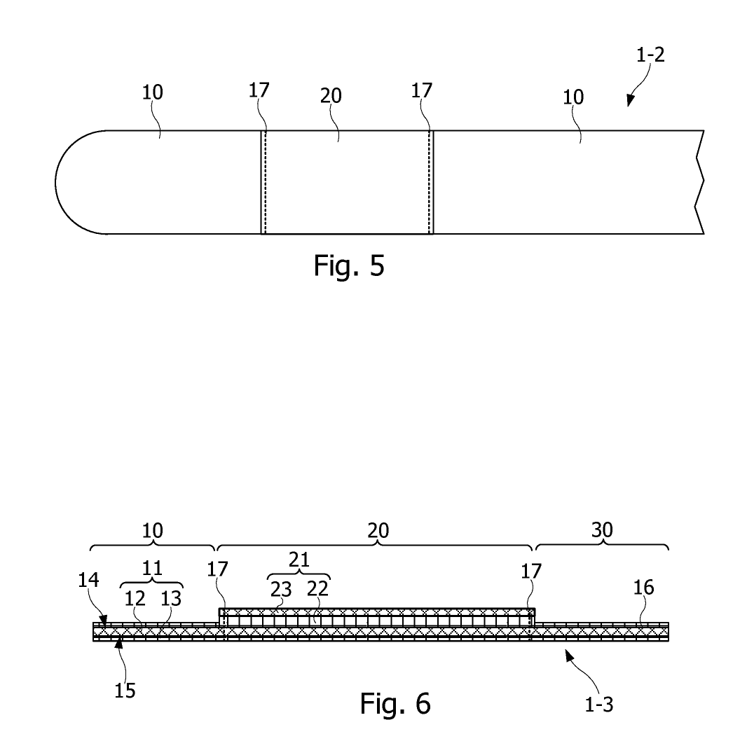

FIGS. 4 and 5 show a supporting device 1-2 according to an embodiment. The supporting device 1-2 is in the form of an elastic band including elastic band segments 10, 20, 30 having different stiffness values. The segments 10, 30 are formed by an elastic band 11. The segment 20 is created by attaching a pad 21 to a layer 13 of the band 11, by two fastening lines 17 extending along two opposite edges of the pad 21. Thus, the band segment 20 has a multilayer structure including the band 11 and the pad 21. The fastening lines 17 are formed on the band 11 along a transverse direction of the band 11. The fastening lines 17 are formed so that the pad 21 between the two fastening lines 17 has a length corresponding substantially to that of the portion of the band 11 under the pad between the two fastening lines 17, in a configuration where the band 11 and the pad 21 are not elongated.

The band 11 may include a layer 12 of elastic material on its outer face 14 and an elastic layer 13 of a material having the desired adhesion on its inner face 15. The layer 12 may be made of fabric that is elastic at least in the longitudinal direction of the band. The layer 13 may be a coating of a viscoelastic material distributed on the inner face 15, for example in a uniform manner. The fastening lines 17 may be seams. The pad 21 may include a layer 22 of a viscoelastic material forming the adhesive surface of the segment 20, and a layer of elastic fabric 23 attached to the layer 22. The fabric layer 23 may be glued to the layer 22. The layer 22 has a certain adhesion preventing it from sliding over the surface covered by the device 1-2, in the presence of a certain compression pressure. The adhesion is such that the band segment 20 can be elongated under the effect of a stretching force different from that applied to the segments 10, 30, and retain its elongation in the absence of the stretching forces, thanks to the adhesion of the layer 22 and the compression pressure exerted by the supporting device 1-2.

The thickness of the band 12 may be chosen between 0.3 and 1.5 mm, the layer 13 having a thickness between 0.1 and 0.5 mm. The layers 2, 13 and 22 can be viscoelastic polymer gels such as silicone gels (Polydimethylsiloxane—PDMS) whose hardness and adhesion properties are chosen according to their respective roles in the supporting device. The thickness of the layer 22 may be chosen between 0.3 and 1 mm.

In one embodiment, the band 10 has a width between 5 and 6 cm, while the pad 21 is slightly narrower than the band 10, for example by about 2 to 7 mm.

Since the adhesive layer 13 is distributed over the entire surface of the band 12, the pad 21 can be fixed to the outer face 14 of the band 12, as illustrated in FIG. 6. Thus, FIG. 6 shows a supporting device 1-3 which differs from the supporting device 1-2 only in that the pad 21 is fixed on the outer face 14 of the band 11. As a result, the layer 13 formed on the inner face of the band 11 is entirely in contact with the area covered by the supporting device 1-3.

The supporting device 1, 1-1, 1-2, 1-3 can be kept wound and stretched around a limb or a joint, with the aid of an attachment device including two parts respectively fixed at the ends of the band 10 and cooperating with each other to attach to one another. This attachment device may for example include one or more loops attached to one end of the supporting device and hooks attached to an opposite end of the supporting device, or hook and loop bands (Velcro®) attached to the opposite ends of the supporting device.

Thus, the supporting device 1, 1-1, 1-2, 1-3 can be wound around a limb or joint by forming more than one turn around the limb, so that part of the inner face 15 of the supporting device is in contact with part of the outer face 14 of the supporting device. It can then be provided to cover at least part of the outer face 14 of the supporting device with an adhesive layer 16 (FIG. 6) which may be made of the same material as the adhesive layer 13. The portion of the outer face 14 covered with the layer 16 may be limited to the portion covered by the outer face 15 of the device, due to the winding of the device over more than one turn. Of course, the layer 16 increases the stiffness of the different band segments where it is present.

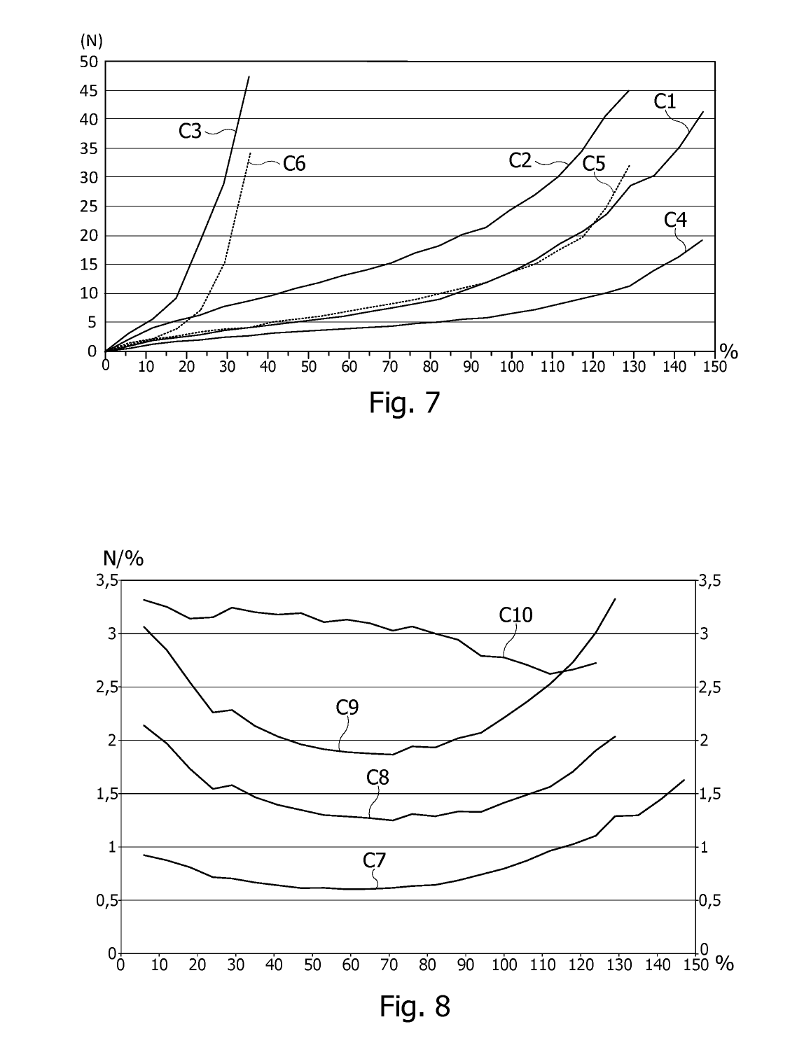

FIG. 7 shows curves C1 to C6 representing the elasticity of elements forming the supporting device 1-2 or 1-3. Each of the curves C1 to C6 illustrates the variations of the stretching force applied to the element as a function of an elongation percentage (in abscissa) of the element. The curve C1 represents the longitudinal elasticity of the band 11. The curve C2 represents the longitudinal elasticity of the pad 21. The curve C3 represents the transverse elasticity of the pad 21. Curve C4 represents the longitudinal elasticity of the band 12 alone. The curve C5 represents the longitudinal elasticity of the fabric layer 23 of the pad 21. The curve C6 represents the transverse elasticity of the fabric layer 23 of the pad 21. The curves C1 to C6 are substantially hyperbolas passing through the origin (coordinates 0%, 0 N). The curves C1 to C4 also have a region between 0 and 80% where the elongation is substantially proportional to the elongation force exerted.

As illustrated by the curves C1 and C2, the band 11 elongates by about 147% and the pad 21 elongates by about 129%, under the effect of stretching forces respectively of 41 N and 45 N. The curves C1 and C2 show in particular that between stretching force values of 5 and 40 N, the band 11 extends 23 to 43% more than the pad 21. According to the curve C3, the pad 21 elongates in the transverse direction by 35% under the effect of a stretching force of 47.5 N. The pad 21 therefore has in the transverse direction a lower elasticity than in the longitudinal direction (curves C2, C3). This arrangement prevents the edge and center regions of the pad 21 from stretching with different elongations. This difference in stiffness is conferred by the fabric layer 23 forming the pad 21. Indeed, according to the curves C5 and C6, the fabric layer 23 extends in the longitudinal direction by 124% and in the transverse direction by 34%, under the effect of a stretching force of 32 N. As illustrated by curve C4, the band 11 alone elongates by 147% under the effect of a stretching force of 19.5 N.

According to an embodiment, the fabric layer 23 has a stiffness in the direction of its attachment to the band 11, two to four times greater in the transverse direction than in the longitudinal direction of the band 11.

FIG. 8 shows stiffness curves C7, C8, C9, respectively for the band segments 10, 30 (band 11) of the supporting device 1-2 or 1-3, of the pad 21 alone, and of the segment 20 combining the band 11 and the pad 21. According to the curve C7, the stiffness of the segments 10, 30 varies between 0.6 and 1.25 N/% when the elongation of these segments is between 5 and 130%. According to the curve C8, the longitudinal stiffness of the pad 21 varies between 1.25 and 2.1 N/% when the longitudinal elongation of the pad 21 is between 5 and 130%. According to the curve C9, the stiffness of the band segment 20 (pad 21+band 11) varies between 1.8 and 3.3 N/% when the elongation of the band segment is between 5 and 130%.

FIG. 8 also shows a curve C10 representing the variation of the stiffness ratio of the band segment 20 and the segment 10 or 30 of the supporting device 1-2 or 1-3. This ratio varies between 2.6 and 3.3 for elongations varying between 5 and 130%. The stiffness of the band segment 20 is therefore on average three times greater than the stiffness of the band segments 10 and 20 in the example of the supporting device 1-2 or 1-3. Due to the presence of the segments 10, 30, the supporting device can adapt to different limb or joint sizes without having to significantly increase its traction around the limb or joint, while having fixed attachment points at its ends. In contrast, the band segment 20 can withstand a high traction without elongating significantly. It can thus impart relatively high pressures to the area it covers. Thus, in the example of FIGS. 7 and 8, the band segment 20 extends about three times less than the band segment 10 or 30 under the effect of the same traction force.

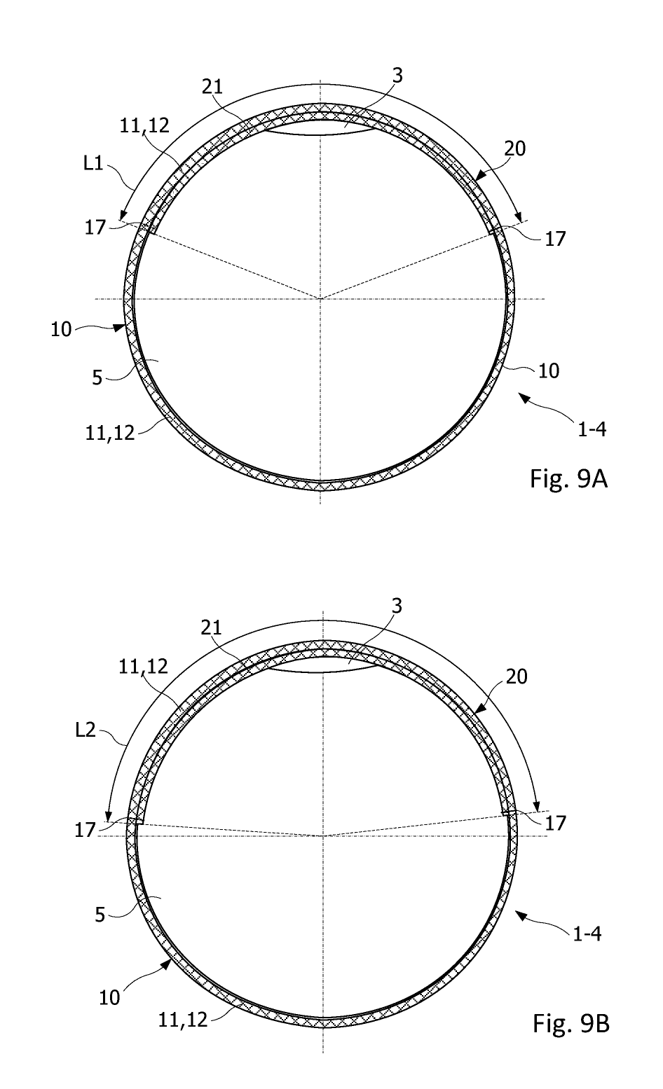

According to an embodiment, the supporting device is configured in the form of a sleeve by attaching together the two opposite ends of the band 11, as illustrated in FIGS. 9A, 9B.

FIGS. 9A, 9B show a supporting device 1-4 in two different configurations, to illustrate a use case of the supporting device. It should be noted that these two configurations can be obtained similarly with any of the supporting devices 1, 1-1 to 1-3 in the form of a band, as soon as it is wound around a limb or joint and attached. In the example of FIGS. 9A, 9B, the supporting device 1-4 includes two band segments 10, 20, the band segment 20 having a greater stiffness than the band segment 10. In FIG. 9A, the supporting device 1-4 has been fitted around a limb or joint 5 with uniform tension around the limb. In the configuration of FIG. 9A, the band segment 20 has a length L1. In this configuration, the supporting device 1-4 exerts on the skin a certain compression force which is substantially constant all around the limb or joint 5, and which is linked to the cumulative elongation of the band segments 10 and 20 given the different stiffness values of the two band segments 10, 20.

In FIG. 9B, the supporting device has been fitted around the limb or joint by applying to the band segment 20 a traction force greater than the traction force applied to the band segment 10. In the configuration of FIG. 9B, the band segment 20 reaches a length L2 greater than the length L1. This configuration is stable to a certain extent in the absence of traction force, due to the adhesion of the inner face of the band segment 20 with the area covered by the supporting device 1-4, appearing in the presence of the compression pressure exerted by the device 1-4. The configuration of the device 1-4 in FIG. 9B can be obtained from that of FIG. 9A, for example by holding with one hand each end of the band segment 20, and further stretching this band segment around the limb 5. As a result, in the configuration of FIG. 9B, the band segment 10 has an elongation smaller than that of the configuration of FIG. 9A, and therefore it exerts less pressure.

In the configuration of FIG. 9B, the band segment 20 exerts on the area of the member that it covers, a greater compression pressure than that exerted by the band segment 10 in the configuration of FIG. 9A or 9B. Furthermore, the band segment 10 in the configuration of FIG. 9B exerts on the area of the member that it covers, a lower compression pressure than that exerted by the band segment 10 in the configuration of FIG. 9A. The supporting device can thus be fitted around a limb or a joint so as to exert lower compression pressure in a certain area covered by the band segment 10, and higher pressure in another area covered by the band segment 20.

According to an embodiment, the supporting device 1-4 includes a cushion 3 of a viscoelastic material attached to the inner face of the device, designed to contact the area covered by the device. In the example of FIGS. 9A, 9B, the cushion 3 is attached to the inner face of the pad 21. The cushion 3 is designed to exert a higher pressure in a localized area. The cushion 3 may have a shape adapted to the area of the skin where a higher pressure should be applied. The position of the cushion 3 may be chosen by the user. For this purpose, the cushion 3 and the pad 21 may be silicone gel (PDMS), having the property of adhering naturally in a detachable manner to another element in the same material.

The sleeve 1-4 shown in FIGS. 9A, 9B may be designed to help strengthen the connection between the periosteum and the tibia bone. For this purpose, the band segment 20 is placed on the tibia, while the band segment 10 covers the rear part of the leg consisting of muscles and tendons that should not be compressed. By stretching the band segment 20 under the effect of a greater traction force than that applied to the band segment 10, the device 1-4 exerts a higher pressure localized on the anterior region of the leg, while exerting low pressure on the muscle mass at the back of the leg. The pressure exerted on the anterior region of the leg favors support of the tibia-periosteal region, which can be highly solicited, in particular during the practice of sports, subjecting this part of the body to repeated intense vibrations and intense tractions applied in particular to the muscle insertions present in this part of the body. The sports involved include running, dancing, and racket sports such as tennis and squash. The presence of the slacker band segment 10 is particularly important because it avoids a tourniquet effect on the calf which swells strongly when it is solicited.

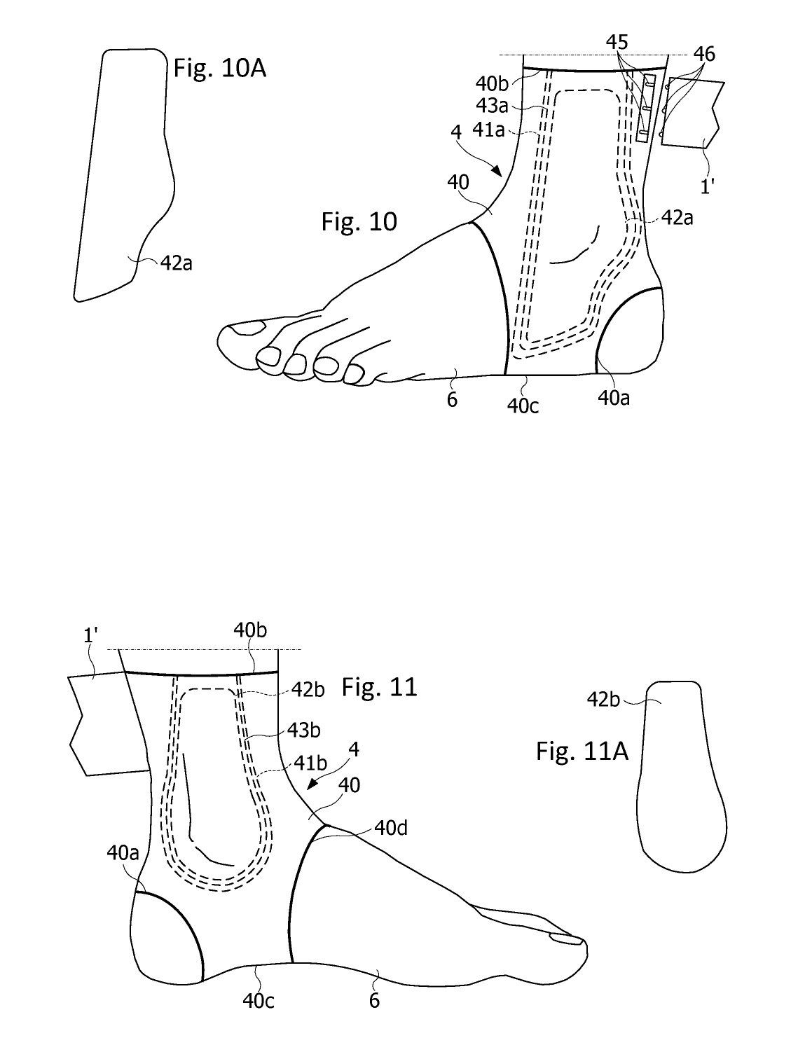

FIGS. 10 and 11 show an orthosis 4 adapted to supporting the ankle, for example following a trauma, or having laxity justifying a formal support or support from a proprioceptive point of view. The orthosis 4 includes an at least partially elastic layer 40, forming a sleeve shaped to compress the ankle and the foot 6, having a proximal opening 40b for the penetration of the foot and the leg, a distal opening 40d for the exit of the foot 6, and an intermediate opening 40a for the heel. The layer 40 is at least partially elastic. A layer 41a is attached to the layer 40 so as to form a first pocket in which a first pad 42a of a thermoformable material is inserted. The shape of the first pocket conforms to the shape of the pad 42a. Thus the degree of filling of the pocket by the pad may be between 80 and 95%. The shape of the pad 42a may itself be defined according to an area of the limb or joint where support is to be ensured. The first pocket is formed on the layer 40 at a location corresponding to the external malleolus and extends laterally between the openings 40a and 40d and between the proximal opening 40b of the layer 40 and a portion 40c of the layer 40 covering a lateral edge of the foot 6 between the plantar and dorsal zones. The shape of the pad 42a is shown in FIG. 10A. The first pocket is formed by attaching the layer 41a to the layer 40, by means of a fastening line 43a. The first pocket has, for example, an opening along the proximal opening 40b of the sleeve 40, for inserting or removing the pad 42a from the pocket. The layer 40 may be in direct contact with the skin 6 of the user. The layer 41a can be attached to the layer 40 by a weld line or a seam 43a, on one or the other of the faces of the layer 40.

According to an embodiment, the orthosis 4 includes a second pad 42b made of a thermoformable material placed in a second pocket formed on the layer 40 at a location configured to cover the internal malleolus and extending laterally on the layer 40 around the internal malleolus to the opening 40b of the sleeve. The shape of the pad 42b is shown in FIG. 11A. The second pocket 42b is formed using a layer of fabric 41b attached to the sleeve 40 by a fastening line 43b. The second pocket may also have an opening along the proximal opening 40b of the sleeve 40 to insert or remove the pad 42b from the pocket.

It should be noted that FIGS. 10A and 11A show the shapes of the pads 42a, 42b before they are thermoformed directly on the foot. After the thermoforming operation, the pads 42a, 42b take the exact shapes of the malleoli and the surrounding areas.

A band 1′ (part of which is shown in FIGS. 10, 11) designed to be wound and tightened around the foot 6 and the ankle, can be used to further tighten the pads 42a, 42b against the ankle, and so make the pads 42a, 42b integral with the ankle. The band 1′ can be sewn or hooked to the orthosis 4 by an attachment device comprising, for example, one or more hooks 45 on the orthosis 4, each cooperating with a loop 46 in an end region of the band 1.

The orthosis 4 can be used in the following manner. Before a first use, the orthosis should undergo a thermoforming operation. For this purpose, it is heated, for example by immersing it with the pads 42a, 42b in their respective pockets, in hot water at a temperature sufficient to soften the pads 42a, 42b. A certain time after their contact with the hot water, the pads 42a, 42b become soft. Before the pads 42a, 42b regain their rigidity, the user puts the orthosis 4 around the ankle to support. The elastic traction of the layer 40 presses the pads 42a, 42b against the skin of the user. The pads 42a, 42b then take the shapes of the areas they are pressed against, and harden after a few minutes. During the hardening of the pads 42a, 42b, the ankle is held in the desired final position. The orthosis 4 is thus adapted to the morphology of the location to which it is applied, by thermoforming the pads 42a, 42b, simply by fitting the orthosis on the ankle, the elastic forces exerted by the layer 40 ensuring the holding and the deformation of the previously softened pads 42a, 42b. This operation does not require the intervention of another person and in particular of a professional.

Before the thermoforming operation, the portion of the limb or joint to be supported may be covered with a film, such as a plastic film, to ease the removal of the wet orthosis at the end of the thermoforming operation.

The pads 42a, 42b are, for example, made of a material such as “Aquaplast” manufactured by Patterson. They have a thickness between 1.5 and 5 mm, for example about 2.4 mm. This material becomes soft at 65-75° C. and remains malleable for about four minutes. Thus, the thermoforming operation of pads 42a, 42b can be repeated as many times as necessary.

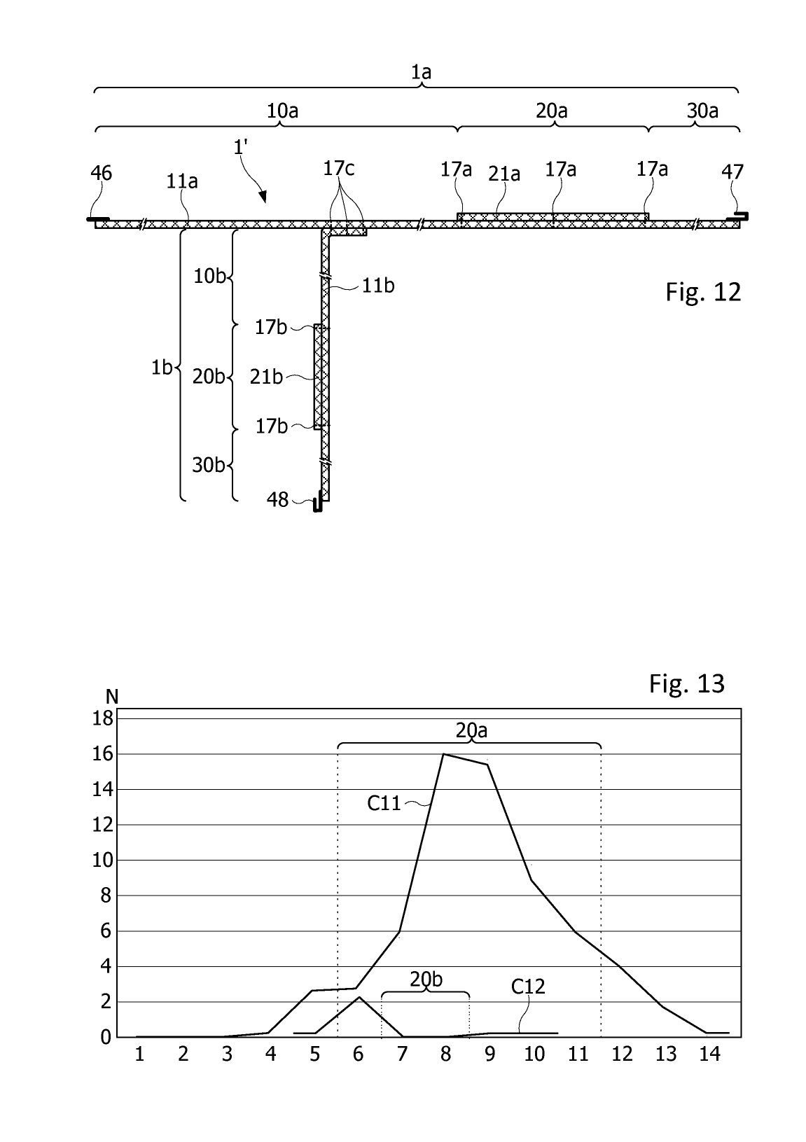

FIG. 12 shows an exemplary embodiment of the band 1′ that can be attached to the ankle orthosis 4. The band 1′ is configured to wrap around the ankle and foot 6. For this purpose, the band 1′ comprises a main portion 1a and a secondary portion 1b fixed to the main part 1a, the band 1′ thus having a general T-shape. The band portion 1b partially covers the part 1a, and thus strengthens the support achieved by the latter. The band portions 1a, 1b have a structure similar to that of the device 1. In the example of FIG. 12, the band portions 1a, 1b have the same structure as device 1-2. Thus, the main part 1a includes band segments 10a, 20a, 30a of different stiffness values, such as the band segments 10, 20, 30, a pad 21a similar to the pad 21, forming the band segment 20a with the band portion 1a. The pad 21a is attached to the inner face of the band portion 1a, for example by seams 17a. The secondary band portion 1b also includes band segments 10b, 20b, 30b of different stiffness values, such as the band segments 10, 20, 30, a pad 21b similar to the pad 21, forming the segment 20b with the band portion 1b. The pad 21b is attached to the inner face of the band portion 1b, for example by seams 17b. One end of the band portion 1b is attached, for example by seams 17c, to the outer face of the band portion 1a opposite the inner face where the pad 21a is attached.

One end of the band portion 1a is first attached to the orthosis 4, for example by means of loops 46 and hooks 45 located on an upper part of the orthosis in the lower region of the calf. The band 1′ can also be sewn to the orthosis 4. In a first step, the band portion 1a is wound without traction around the ankle over a little more than half a turn passing behind the ankle, then around the foot over the top of the foot, and finally under the foot. Then, the user can place his foot on the ground so as to hold in place one end of the segment 20a of the band portion 1a, then wind the segment 20a while exerting a stronger traction on the free end of the band portion 1a. The segment 20a is then slightly elongated while covering a lower portion of the pad 42a that is pressed against the foot. Then, moving back up, the band covers the upper part of the arch of the foot, insensitive to pressure, avoiding the lower and very sensitive part of the arch, where it could hinder the dorsal flexion of the foot. Finally, the segment 20a covers most of the pad 42b (and in particular the portion of the pad 42b covering the internal malleolus). Once the segment 20a is wrapped around the ankle, the traction on the free end of the band portion 1a is released and its free end is attached near the loops 46 by another hook and loop device 47 provided for this purpose. Due to the adhesion of the inner face of the segment 20a, it maintains its elongation, especially while the end of the band 1a is attached. The band segment 20a thus creates a strong connection between the two pads 42a, 42b.

The end of the band portion 1b attached to the band portion 1a is then parallel to the underside of the foot on the inner side face of the foot. The band portion 1b is wound without traction around the ankle over a little more than a half-turn over the foot, then behind the ankle, so that the pad 21b is in contact with the band portion 1a. The free end of the band portion 1b is then attached to the band portion 1a near the loops 46, by means of another attachment device, for example hooks and loops 48. In this configuration of the band portion 1b, the band segment 20b covers the upper portion of the pad 42a, holding it tight against the ankle.

FIG. 13 shows curves C11, C12 illustrating the variation of the traction force of the band 1′ after it has been wound around the foot and the orthosis, in locations along the band. The band segments 1a, 1b were divided into numbered sections of identical lengths before stretching, the section numbers being presented on the abscissa. Thus, in FIG. 13, the band portion 1a corresponding to the curve C11 extends over the sections numbered from 1 to 14, while the band segment 20a extends over the sections numbered from 6 to 11. The band portion 1b corresponding to the curve C12, is attached to the band portion 1a between the sections bearing the numbers 4 and 5, and extends over the sections numbered 5 to 10, and the band segment 20b extends over the sections numbered 7 and 8.

According to the curve C11, the band portion 1a is wound without traction up to the section number 3. Up to the section number 4, the traction force exerted by the band portion 1a increases slightly from 0 to 0.2 N. This traction force increases further to reach 2.72 N at the section number 6 corresponding to the beginning of the segment 20a. The section 20a exerts a traction force which progressively increases to reach a value close to 16 N in the vicinity of the section numbers 8 and 9. The traction force exerted by the band portion 1a then decreases progressively to reach a value close to zero at the end of the band 1a at section number 14. At the junction with the band segment 20a, the band segment 10a reaches an elongation rate of about 15%. The elongation of the band segment 20a progressively increases from 8 to 45% between the section numbers 6 and 8, then decreases to reach 15% at section number 11. The elongation of the band segment 30a decreases progressively from 25% to 5% between the section numbers 12 and 14.

According to the curve C12, the band portion 1b is wound to reach a slight traction force of 0.2 N which increases up to 2.25 N at the section number 6. This traction force is zero or close to zero along the band segment 20b, and reaches 0.2 N at the end of the band portion 1b at section number 10. The elongation of the band segment 10b increases from 5 to 13% between the section numbers 5 and 6. The elongation of the band segment 20b is zero, and the elongation of the segment 30b is 5%.

When the band is wrapped around the orthosis and the foot, the section number 9 (middle portion of the pad 21a) covers the sections 3 and 4 of the band portion 1a. The sections numbers 7 and 8 (pad 21b) cover the sections numbers 9 and 14 of the band portion 1a. The section number 9 of the band portion 1b covers the section number 12 of the band portion 1a. The compression forces exerted by the different superimposed segments add up.

Since the band segment 20b is not fitted with an elongation, the pad 21b can be made of a non-elastic material. Along the band 1′, the conservation of the different traction forces exerted, which can differ by 20 N, is ensured by the adhesion of the band on itself and on the orthosis.

The segments 20a, 20b of the band 1′, which are tighter and of higher stiffness, thus hold the pads 42a, 42b tight against the ankle as would an ankle splint, the segment 20b complementing the action of the segment 20a to support the ankle in three points, namely at the bottom and top of the pad 42a and at the pad 42b. The band 1′ does not exert too much pressure on the arch of the foot and on the lower tibial zone just above the malleoli, which are covered by the slacker and suppler segments 10a, 30a, 10b, 30b. In addition, the segments 10a, 30a, 10b, 30b can be stretched more or less to appropriately place the segments 20a, 20b over the pads 42a, 42b depending on the morphology of the user, without significant variation in the pressure that they exert on the areas of the ankle they cover.

The ends of the band portions 1a, 1b supporting the loops and the hooks 45 to 47 may be reinforced by pieces of non-elastic fabric.

It will be apparent to those skilled in the art that the present invention is susceptible of various alternative embodiments and various applications. In particular, the invention is not limited to the embodiments described, but extends to all possible combinations of these embodiments.

Furthermore, the adhesion of the inner face 15 of the device 1, 1-1, 1-3, or 1-4, the band 11 of the device 1-2, or the band 1′ can be obtained by viscoelastic elements such as studs, distributed on the face 15, for example in a uniform manner. The density of these viscoelastic elements on the inner face 15 may be chosen as a function of the adhesion to be obtained taking into account the compression pressure exerted by the device and the adhesion of each viscoelastic element.

Although the described examples apply to securing the periosteum and supporting the ankle, the invention may be applied to any part of the body requiring selective application of pressure in one or more distinct areas.

1-12. (canceled)

13. A supporting or compressing device configured to surround a limb or a joint of a user, comprising an elastic band having two band segments having different stiffness values, the elastic band having an inner face in contact with an area covered by the band and an outer face opposite to the inner face, the inner face of the band providing, in the presence of a compression pressure exerted by the band when it is fitted under traction around a limb or joint, an adhesion with the covered area, such that the band conserves locally elongations resulting from different traction forces, after removal of the traction forces.

14. The device according to claim 13, wherein a first of the two band segments comprises a pad made of an elastic material, attached by two opposite ends to the band.

15. The device according to claim 14, wherein the pad is attached to the inner face of the supporting device.

16. The device according to claim 14, wherein the pad is attached to the outer face of the supporting device.

17. The device according to claim 13, wherein the pad comprises a layer of viscoelastic material and a layer of elastic fabric attached to the layer of viscoelastic material.

18. The device according to claim 17, wherein the elastic fabric layer has a lower stiffness in a longitudinal direction of the band than in a transverse direction.

19. The device according to claim 13, wherein a first of the two band segments has a stiffness between 2 and 4 times that of a second of the two band segments.

20. The device according to claim 13, wherein the inner face is formed by a layer of a polymer gel such as Polydimethylsiloxane.

21. The device according to claim 13, wherein the elastic band is configured in the form of a sleeve.

22. The device according to claim 13, wherein the inner face of the band has, in the presence of a compression pressure exerted when the band is placed under traction around a member or a joint, an adhesion with the covered area which can retain elongations resulting from stretching forces that differ by 20 N.

23. The device according to claim 13, comprising first and second band portions configured to be wound around the ankle and foot, the first band portion including a first band segment having a higher stiffness than another band segment of the first band portion, the second band portion having an end fixed to the outer face of the first portion, a second band segment having a higher stiffness than another band segment of the second band portion, wherein the first and second band segments of higher stiffness are arranged on the band portions so that they can simultaneously cover the internal malleolus and regions below and above the external malleolus of the ankle.

24. A method of manufacturing a supporting or compressing device configured to surround a limb or joint of a user, according to claim 13, the method comprising steps of assembling elastic band segments to obtain band segments of different stiffness values.
